(19) United States
(12) Patent Application Publication
(10) Pub. No.: US 2020/0114031 A1
(43) Pub. Date: Apr. 16, 2020

(54) DEVICE FOR TREATING SYSTEM COMPONENTS AND/OR PACKAGING MEANS USING VAPORIZED HYDROGEN PEROXIDE

(71) Applicant: KRONES AG, Neutraubling (DE)

(72) Inventors: Gertrud SOELLNER-WEIN, Neutraubling (DE); Juergen SOELLNER, Neutraubling (DE); Holger MUELLER, Neutraubling (DE)

(21) Appl. No.: 16/470,485

(22) PCT Filed: Dec. 20, 2017
(86) PCT No.: PCT/EP2017/083725

§ 371 Date: Jun. 17, 2019

(30) Foreign Application Priority Data
Dec. 20, 2016 (DE) 10 2016 125 027.9

(51) Int. Cl. A61L 2/24; A61L 2/20; B65B 55/10

(52) U.S. Cl. CPC A61L 2/24; A61L 2/208; B65B 55/10; A61L 2202/23; A61L 2202/15; A61L 2202/14; A61L 2202/11

(57) ABSTRACT

A device for treating system components and/or packaging means in a filling product filling system, comprising a vaporization device for providing a treatment gas flow containing vaporized hydrogen peroxide, further comprising a treatment nozzle for applying the treatment gas flow to the system components to be treated and/or the packaging means to be treated, wherein a sensor is provided for measuring the hydrogen peroxide concentration in the treatment gas flow exiting the treatment nozzle.

### TECHNICAL FIELD

The present invention relates to a device for treating system components and/or packaging means using vaporized hydrogen peroxide, in particular for treating beverage containers with vaporized hydrogen peroxide in order to sterilize them before the sterilized containers are filled with a filling product in a beverage filling system.

### TECHNICAL BACKGROUND

It is known in beverage filling systems to sterilize the packaging means, and in particular the containers that are to be filled with the beverage or filling product, prior to the actual filling. For this purpose it is known to impinge the applicable packaging means, and in particular the beverage containers, with vaporized hydrogen peroxide, which is caused to flow together with a stream of carrier gas onto the packaging means and/or into the packaging means. In this context it is known to move the packaging means, for example by means of a treatment carousel, and then blow the vaporized hydrogen peroxide together with the carrier gas into the packaging means via suitable treatment nozzles.

It is further known to use vaporized hydrogen peroxide to sterilize system components of the beverage filling system. In particular it is known to use vaporized hydrogen peroxide to sterilize the interior of isolators and the components of the filling system that are disposed in the isolator by impinging them with vaporized hydrogen peroxide. It is additionally known to impinge the channels in a beverage filling system that come into contact with the filling product with vaporized hydrogen peroxide, in order to achieve a corresponding sterilization of these surfaces.

The vaporized hydrogen peroxide is also used for treating container closures, for example container caps that can be screwed onto filled containers. In this case it is known to impinge the container closures with the mixture of vaporized hydrogen peroxide and carrier gas inside a treatment tunnel, through which the container closures are conveyed, and within which an atmosphere formed from the carrier gas and the vaporized hydrogen peroxide is present.

In particular in dry aseptic systems, the packaging means is sterilized by means of vaporized hydrogen peroxide. In this case heated air, with a temperature of up to 130° C., is used as a carrier medium, i.e. a carrier gas, and the vaporized hydrogen peroxide is conveyed together with the carrier gas via a rotary distributor to the treatment nozzles on the applicable treatment carousel. In this manner both the packaging means and, for example, preforms can be impinged with the vaporized hydrogen peroxide.

For this purpose the hydrogen peroxide is usually supplied in liquid form—for example as an aqueous hydrogen peroxide solution—to a vaporization device, in which it is vaporized. It is then transported, by a flow of carrier gas that passes over the vaporization device, together with the carrier gas to the applicable treatment location as a treatment gas. The concentration of vaporized hydrogen peroxide in the flow of carrier gas and vaporized hydrogen peroxide can be determined from the knowledge firstly of the volume flow of the carrier gas through the vaporization device and secondly of the quantity of liquid hydrogen peroxide that is supplied to be vaporized. From these two parameters it is possible to calculate the concentration of hydrogen peroxide in the flow of carrier gas and hydrogen peroxide leaving the vaporization device.

Determining the concentration of vaporized hydrogen peroxide in the flow of carrier gas and hydrogen peroxide is important in order to ensure reliable sterilization.

Because, however, the vaporizer for vaporizing the hydrogen peroxide is usually at some distance from the actual point of use, the concentration of vaporized hydrogen peroxide in the flow of carrier gas can be calculated directly downstream of the vaporizer but not at the location at which it is actually used. The hydrogen peroxide is already subject to decomposition in the piping system on the way between the vaporizer and the point of use. This decomposition depends, for example, on the length of the piping, the material of the piping, the temperature, and any contamination that may already be present in the piping system.

Furthermore, the calculation of the concentration does not absolutely ensure that the calculated concentration corresponds to the actual concentration. This is because both the concentration of hydrogen peroxide in the aqueous hydrogen peroxide solution and the volume flow of the carrier gas can vary. Thus in known systems, in order to be able to provide reliable sterilization, a higher concentration of hydrogen peroxide is required in the flow of carrier gas and hydrogen peroxide from the vaporizer, as a “safety margin” to compensate for any deviations between the calculated concentration and the actual concentration at the point of use. As a result, the consumption of hydrogen peroxide can be higher than that which is necessary to achieve reliable sterilization.

### SUMMARY OF THE INVENTION

Accordingly, an object of the present invention is to propose a device for sterilizing system components and/or packaging means using vaporized hydrogen peroxide which provides an improved design.

This object is achieved by a device with the features of claim 1. Advantageous further developments arise from the dependent claims, the figures and the present description.

Accordingly, a device for treating system components and/or packaging means in a filling product filling system is proposed, comprising a vaporization device for providing a treatment gas flow containing vaporized hydrogen peroxide, further comprising a treatment nozzle for applying the treatment gas flow to the system components to be treated and/or the packaging means to be treated. According to the invention, a sensor is provided for measuring the hydrogen peroxide concentration in the treatment gas flow exiting the treatment nozzle.

Due to the fact that a sensor is provided to measure the hydrogen peroxide concentration in the treatment gas flow exiting the treatment nozzle, a precise measurement of the hydrogen peroxide concentration in the treatment gas can be carried out directly at the location of treatment. In this manner it is possible to evaluate the level of sterilization at the treatment location, so that reliable sterilization of the applicable packaging means and/or system components can be achieved.

It is also possible by this means to dispense with verification of the hydrogen peroxide concentration by an external measurement to ensure the specified level of sterilization. Because the hydrogen peroxide concentration can be measured directly at the location of treatment, there is no longer the uncertainty as to the actual hydrogen peroxide concentration at the treatment location that exists in conventional devices due to the estimates that are made. Instead, only the hydrogen peroxide concentration at the location of treatment is taken into account.

Furthermore, due to the fact that a sensor is provided to measure the hydrogen peroxide concentration at the location of treatment, it is possible to control or regulate the vaporization device for vaporizing the liquid hydrogen peroxide such that the hydrogen peroxide concentration in the resulting flow of hydrogen peroxide and carrier gas, i.e. the treatment gas flow, reaches the desired value, irrespective of the losses that the hydrogen peroxide has suffered in the upstream piping system. Losses of the hydrogen peroxide in the piping system upstream of the treatment nozzle, and hence a reduction in the hydrogen peroxide concentration, can occur due to decomposition of the hydrogen peroxide. The decisive factors contributing to this decomposition are the temperature and the material of the piping system along with contamination that is present in the piping system. These factors can be excluded by the measurement of the hydrogen peroxide concentration at the location of treatment.

In this manner it can further be ensured that the desired level of sterilization can be achieved with the use of as little liquid hydrogen peroxide as possible. This is because the concentration can be monitored at the treatment location, and the quantity of liquid hydrogen peroxide that must be vaporized is no greater than that which is necessary to achieve the desired hydrogen peroxide concentration. Thus the consumption of hydrogen peroxide can also be minimized or optimized, since the “uncertainty surcharge” no longer applies, i.e. the “safety margin” that was previously necessary to compensate for losses in the piping can be dispensed with. Instead, only the exact amount of hydrogen peroxide that is actually needed at the treatment location is consumed.

The sensor for measuring the hydrogen peroxide concentration is preferably provided in the treatment nozzle and/or on the treatment nozzle, so that no additional component for holding the sensor is required. Furthermore, in this manner the sensor is disposed directly at the treatment location.

The sensor is preferably disposed on a rotating part of the device, in particular on a treatment carousel upon which the treatment nozzle is disposed. The sensor can thereby measure the hydrogen peroxide concentration in the treatment gas that emerges from the treatment nozzle.

In this manner it is possible, also in the case of a rotary treatment device, to monitor precisely the treatment of the packaging means that is to be treated, i.e. its impingement with hydrogen peroxide.

The design of the system as a whole can thereby be simplified. In particular, it is also possible to dispense with the measurement of the concentration of hydrogen peroxide in the liquid hydrogen peroxide. Thus it is possible to dispense with both this sensor and a sensor for measuring the volume flow through the vaporizer, or else such a sensor need not require a very high level of accuracy. The only measurement that remains significant is that of the hydrogen peroxide concentration at the location of treatment, so that in this manner reliable control and/or regulation of the hydrogen peroxide concentration at the treatment location can be achieved.

Preferably, the signal relating to the hydrogen peroxide concentration as measured by the sensor can be transmitted by wire from the rotating part of the device to a stationary part of the device, in particular by means of a slip ring transmitter. In this manner only simple circuitry is required for the transfer of the sensor signal from the rotating part to the stationary part of the device. Thus a measurement of the hydrogen peroxide concentration at the treatment location can also be carried out on a treatment carousel, and the transmission of the measurement signal to the stationary part can take place in a simple manner with respect to its circuitry, in order in this manner to enable the regulation of the supply of liquid hydrogen peroxide to the vaporization device.

In a preferred alternative, the signal relating to the hydrogen peroxide concentration as measured by the sensor is transmitted wirelessly from the rotating part of the device to a stationary part of the device, and the sensor in particular comprises an RFID chip which enables the signal from the sensor to be transmitted to an RFID antenna that is disposed on the stationary part of the device.

In this manner, transmission of the sensors measurement signal from the rotating part to the stationary part can be achieved wirelessly, so that it is possible to dispense with the mechanically complex transfer of the sensor signal from the rotating part to the stationary part using a wired method.

The signal relating to the hydrogen peroxide concentration as measured by the sensor is preferably used for regulating the supply of hydrogen peroxide in the liquid phase into the vaporization device. By this means the consumption of liquid hydrogen peroxide can be optimized, as already described above.

Particularly preferably, the sensor for measuring the hydrogen peroxide concentration is a catalytic sensor, which measures the hydrogen peroxide concentration based on a catalytic decomposition. In such a sensor, the temperature difference between a reference surface and a surface coated with a suitable catalyst is measured, wherein the decomposition of the hydrogen peroxide on the catalytic surface leads to an exothermic reaction that in turn leads to a rise in temperature. The difference between the temperature measured at the reference surface and the temperature of the surface that is coated with the catalyst is a measure of the concentration of the hydrogen peroxide. In this manner, reliable inline measurement of the hydrogen peroxide concentration can take place, so that the hydrogen peroxide concentration can be continuously checked during the operation of the system. It is accordingly no longer necessary to take discrete samples during operation for analysis of the hydrogen peroxide concentration at specific points. Instead, the integration of the catalytic sensor makes continuous monitoring possible. Thus by means of a control or regulation device it is possible using the sensor to control or regulate inline the supply of liquid hydrogen peroxide to the vaporizer and/or vary the flow of carrier gas through the vaporizer.

It is further possible by means of the sensor to carry out a validation of the sterilization of the system, including when it is brought into service or at the beginning of the production process, such that the required or desired hydrogen peroxide concentration can be provided over the entire piping system, and in this manner it can be ensured that the hydrogen peroxide concentration corresponds to the specified concentration of hydrogen peroxide.

The advantageous effects of the device described here are also achieved when it is used for the sterilization of container closures. In this case the provision of a sensor at the location of treatment in particular enables efficient use of the hydrogen peroxide that is present in the liquid phase, such that the hydrogen peroxide is used in no more than the quantities that are actually needed subsequently at the treatment location. An additional quantity in excess of this, which in the prior art is deliberately added as a “safety margin”, is no longer necessary.

The treatment nozzle preferably has an outlet aperture for guiding the flow of treatment gas into a mouth area of a packaging means that is to be treated, and a torispherical head is provided for deflecting the displaced treatment gas that flows out of the mouth area of the packaging means onto the exterior of the packaging means, wherein a sensor is disposed in a first sensor position in the treatment gas supply line immediately before the outlet aperture, and/or a sensor is disposed in a second sensor position in the torispherical head.

By means of the arrangement of the sensor in the treatment nozzle itself, the treatment gas can be particularly reliably monitored as to its hydrogen peroxide concentration.

### BRIEF DESCRIPTION OF THE FIGURES

Preferred further embodiments of the invention are more fully explained by the description below of the figures. The figures show:

FIG. 1 a schematic representation of a device for treating packaging means in a first embodiment, in which a sensor for determining the hydrogen peroxide concentration is positioned at the inlet into an isolator;

FIG. 2 a further schematic representation of a device for treating packaging means, in which a sensor for determining the hydrogen peroxide concentration is positioned in the housing of the isolator;

FIG. 3 a schematic representation of a device for treating packaging means, in which a sensor for determining the hydrogen peroxide concentration is positioned before the point of transfer to a rotary media distributor;

FIG. 4 a schematic representation of a device for treating packaging means, in which a sensor for determining the hydrogen peroxide concentration is positioned on a treatment nozzle of a treatment carousel;

FIG. 5 a schematic representation of a device for treating packaging means, in which a sensor for determining the hydrogen peroxide concentration is disposed on a treatment carousel, and transmits wirelessly to a stationary receiver;

FIG. 6 schematic, perspective and sectional representations of the possible arrangement of a sensor on a treatment nozzle of a treatment device;

FIG. 7 a schematic representation of a treatment carousel; and

FIG. 8 a schematic representation of the design of a sensor.

### DETAILED DESCRIPTION OF EXAMPLES OF PREFERRED EMBODIMENTS

Examples of preferred embodiments are described below with the aid of the figures. In the figures, elements which are identical or similar, or have identical effects, are designated with identical reference signs. In order to avoid redundancy, repeated description of these elements is in part dispensed with.

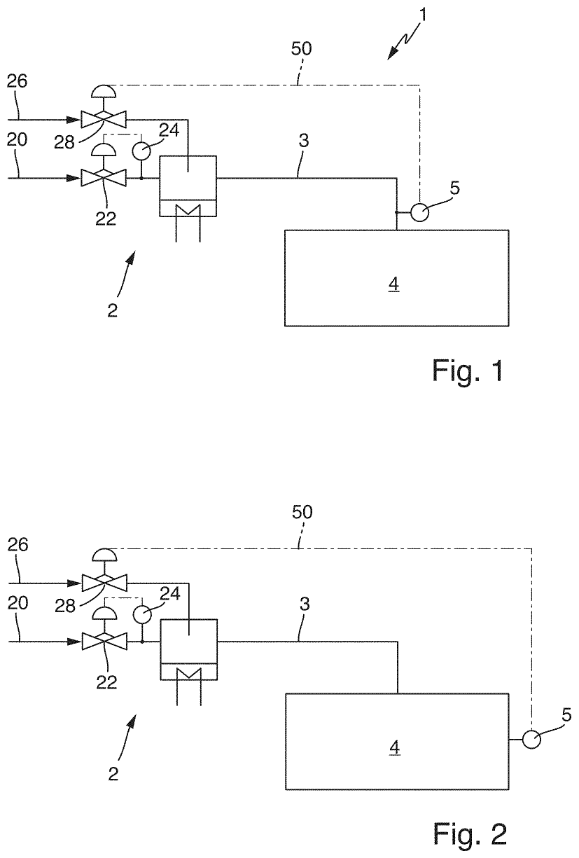

FIG. 1 shows schematically the design of a device 1 for sterilizing system components and treating packaging means. The device 1 comprises a vaporization device 2, by means of which hydrogen peroxide in the liquid phase can be vaporized. The vaporization device 2 is supplied with a carrier gas via a carrier gas supply line 20, wherein the flow of the carrier gas can be regulated by means of a suitable regulating valve 22. A mass flow sensor 24 disposed downstream of the regulating valve 22 can be used for the precise regulation of the mass flow of the carrier gas. The carrier gas that is supplied via the carrier gas supply line 20 thus flows through the vaporization device 2.

As the carrier gas, air, dried air or another gas or gas mixture can for example be used.

The vaporization device 2 is also supplied, via a hydrogen peroxide supply line 26, with liquid hydrogen peroxide, which can for example be supplied in the form of an aqueous hydrogen peroxide solution. By means of a regulating valve 28, it is possible to regulate the supply to the vaporization device 2 of the liquid hydrogen peroxide, which is supplied via the hydrogen peroxide supply line 26.

In the vaporization device 2, liquid hydrogen peroxide is applied to a suitably heated surface, and vaporized on this surface. The vaporized hydrogen peroxide is then entrained and carried away by the carrier gas that is supplied via the carrier gas supply line 20. The flow of treatment gas, which consists of the carrier gas and the vaporized hydrogen peroxide, is then guided out of the vaporization device 2 via a suitable treatment gas supply line 3 to the actual treatment location, in order to treat the system components or packaging means.

In the example embodiment that is shown, an isolator 4 is provided, into which the treatment gas is conveyed via the treatment gas supply line 3 in order to carry out the treatment. In order to measure the concentration of hydrogen peroxide in the treatment gas, i.e. in the volume flow consisting of the carrier gas and the vaporized hydrogen peroxide, a sensor 5 is provided, by means of which the concentration can be measured. The sensor 5 is disposed at the end of the treatment gas supply line 3, and can in this manner measure the hydrogen peroxide concentration of the treatment gas at the end of the treatment gas supply line 3, and thus immediately before, or at the point at which, it enters the isolator 4.

Thus the hydrogen peroxide concentration that is measured by the sensor 5 corresponds to the hydrogen peroxide concentration in the treatment gas which enters the isolator 4. By this means it is possible to ignore potential piping losses within the treatment gas supply line 3, which may have occurred for example due to the decomposition of hydrogen peroxide in the piping between the vaporization device 2 and the point of entry into the isolator 4. These losses can depend on the temperature, the properties and length of the piping, and possibly also any contamination that is present in the treatment gas supply line 3, and thus cannot be precisely predicted. Instead, the precise concentration of hydrogen peroxide at the treatment location—in this case the isolator 4—is known, since it is measured by means of the sensor 5 immediately upstream of the treatment location.

The sensor 5 transmits its concentration signal via a control cable 50 (and possibly via an intermediate regulation and/or control device, which is not explicitly shown here) to the regulating valve 28, by means of which the supply of liquid hydrogen peroxide to the vaporizer 2 is regulated. Thus if the hydrogen peroxide concentration at the sensor 5 deviates from the desired hydrogen peroxide concentration, a volume flow of liquid hydrogen peroxide that is supplied to the vaporization device 2 can be regulated such that the desired hydrogen peroxide concentration is achieved and maintained at the sensor 5, and thus at the treatment location. Thus it is no longer necessary to estimate the losses in the piping and allow an appropriate “safety margin”.

In this manner, the liquid hydrogen peroxide that is supplied via the regulating valve 28 can be efficiently used, and only the amount of liquid hydrogen peroxide that is actually needed at the treatment location, in this case the isolator 4, is vaporized in the vaporization device 2. Thus it is possible to save at least the amount of hydrogen peroxide that was hitherto used to provide a “safety margin” in prior art devices.

Furthermore, by measuring the hydrogen peroxide concentration at the treatment location it can be ensured that correct sterilization according to the specifications is performed in the isolator 4, in that here the appropriate concentration of hydrogen peroxide is reached and the desired and specified sterilization outcome is thereby achieved.

FIG. 2 shows a variant of the device that was shown in FIG. 1. In this case, the sensor 5 is disposed within a wall of the isolator 4, and can in this manner measure the hydrogen peroxide concentration in the treatment gas in the isolator, and thereby appropriately influence the supply of the hydrogen peroxide via the treatment gas supply line 3.

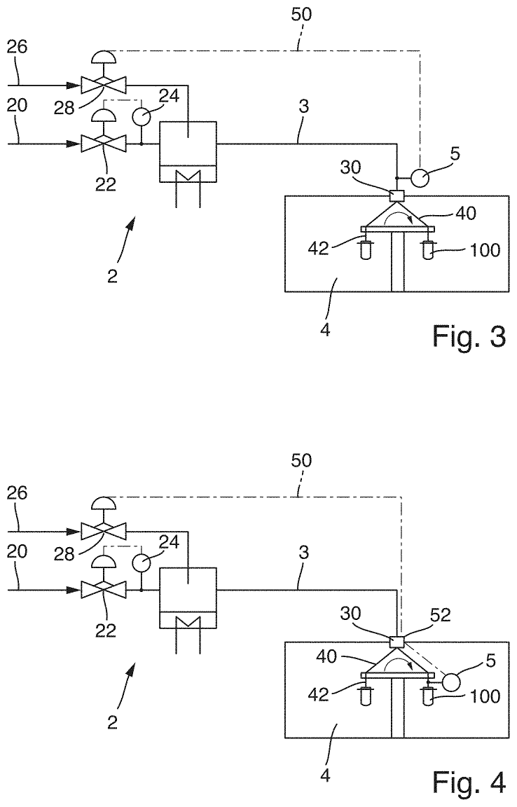

In FIG. 3, a treatment carousel 40 is provided in the isolator 4. In the treatment carousel 40, packaging means 100, which are represented schematically as preforms in the embodiment that is shown, can be sterilized. For this purpose, treatment nozzles 42, by means of which the treatment gas can be blown into the packaging means 100, are provided in the treatment carousel 40, disposed in each case above the packaging means 100. The treatment gas that is supplied via the treatment gas supply line 3 is transferred from the stationary part of the device to the rotating part of the device, i.e. to the treatment carousel 40, by means of a rotary distributor 30.

The sensor 5 is disposed directly before the rotary distributor 30, so that the concentration of the hydrogen peroxide that is transferred to the treatment carousel 40 is known. Here too, therefore, the advantageous effects can be achieved; in particular it can be ensured that the treatment gas that is transferred via the rotary distributor 30 to the treatment carousel 40 has the desired concentration, and therefore that the sterilization outcome within the treatment carousel 40 is not influenced by losses within the treatment gas supply line 3, or fluctuations in the concentration of the hydrogen peroxide in the liquid hydrogen peroxide that is supplied via the hydrogen peroxide supply line 26.

FIG. 4 shows a further variant of the treatment device that was shown in FIG. 3. In this case the sensor 5 is disposed on the treatment carousel 40 itself, and in particular is provided directly on the treatment nozzle 42 or in the treatment nozzle 42. The hydrogen peroxide concentration can thus be measured directly at the treatment location.

In the example embodiment that is shown, the measurement signal relating to the hydrogen peroxide concentration, as measured by the sensor 5, is transferred from the rotary carousel 40 to the stationary part of the device 1 by means of a slip ring transmitter 52, so that the sensor signal from the sensor 5 can then be sent via the control cable 50 to the regulating valve 28. Other types of wired transmission of the measurement signal from the rotating part to the stationary part of the device are also conceivable.

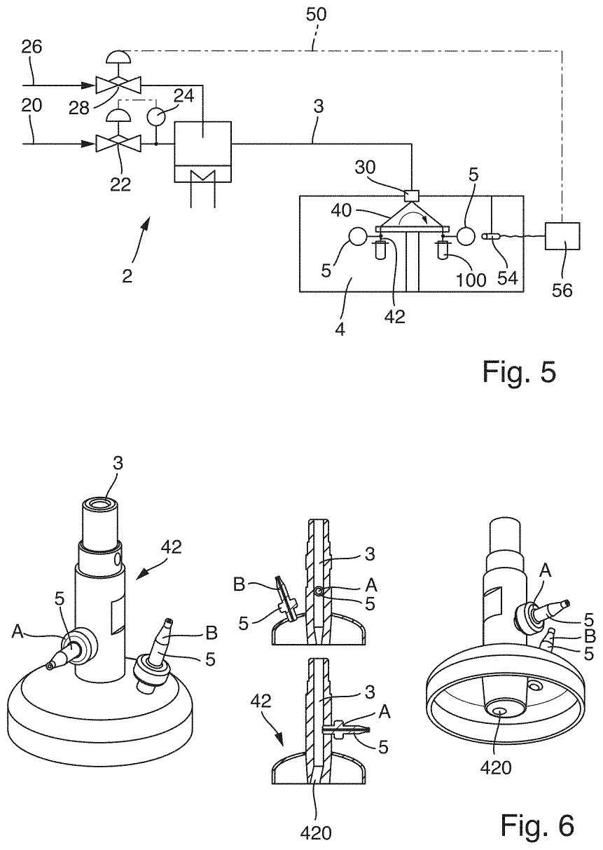

FIG. 5 shows a further variant of the embodiment shown in FIG. 4. Here, the sensor 5 is again disposed on the treatment carousel 40, and thus rotates together with the treatment carousel 40.

In the example embodiment that is shown, the sensor 5 has a wireless transmission device, by means of which the measurement signal can be sent from the rotating part to the stationary part of the device 1.

In the example embodiment that is shown, the sensor 5 has a passive RFID chip, which enables transmission of each sensor signal to an RFID antenna 54 that is disposed on the stationary part of the device. By means of an evaluation device, the RFID antenna 54 passes the sensor signal to the control cable 50, so that by means of the sensor signal the regulating valve 28 for the supply of the liquid hydrogen peroxide can be controlled.

In this example embodiment, due to the equipping of the sensor 5 with a passive RFID chip, it is possible for the sensor signal from sensor 5 to be transferred without further contact from the rotating part of the treatment carousel 40 to the stationary part, i.e. to the RFID antenna 54 that is disposed on the stationary part. The passive RFID chip in the sensor 5 receives energy every time it passes the RFID antenna 54, and this energy is used in the RFID chip to transmit the applicable sensor signal.

Thus in this manner it is possible to achieve the monitoring of the hydrogen peroxide concentration at each individual treatment nozzle 42 upon which a sensor 5 is disposed on the treatment carousel 40. Furthermore, a sensor 5 is thereby provided which can be deployed without additional cabling on the rotating part of the device. In the evaluation device 56, it is then determined which of the plurality of sensor signals that are received is actually to be used to control the regulating valve 28. For this purpose, it is possible to use either a mean value of the sensor values from the sensors 5, or else the lowest concentration measured by the sensors 5.

On the treatment carousel 40, a sensor 5 can be provided on or in every treatment nozzle 42, or only on selected treatment nozzles 42, or even on only one of the treatment nozzles 42. Assuming that the treatment gas supply lines to each treatment nozzle 42 are identical in design, it can be sufficient to use a single sensor 5. If at least one additional sensor 5 is used on another treatment nozzle 42, any sensor errors can be compensated or detected, and any differences in the design of the supply lines to the treatment nozzles 42 can be taken into account.

The provision of sensors on each of the treatment nozzles 42 further enables an appropriate plausibility check to be performed, and makes it possible to ensure that the treatment is carried out to a specified standard on all treatment nozzles 42, and that the packaging means or system components that are treated reach the desired level of sterilization.

The positioning of the sensor 5 on the treatment nozzle 42 is shown schematically for one type of treatment nozzle in FIG. 6. This positioning can in principle also be adopted for other types of treatment nozzle.

In the schematic, perspective and sectional representations in FIG. 6, the sensor 5 is shown positioned in a first position A, in which the sensor 5 is disposed directly in the treatment gas supply line 3 and immediately before the outlet aperture 420 of the treatment nozzle 42. Thus the flow of treatment gas that is measured is precisely the flow that is emitted from the treatment nozzle 42 via the outlet aperture 420. The hydrogen peroxide concentration of the treatment gas that is emitted from the outlet aperture 420 of the treatment nozzle 42 can thereby be precisely measured.

In a second sensor position, which is indicated with the reference sign B, it is possible to measure the hydrogen peroxide concentration of the flow of treatment gas that flows back out of the packaging means after its treatment. The sensor 5 disposed in the second sensor position B is accordingly positioned in the torispherical head. The gas flow that is measured by the sensor 5 in the sensor position B is the gas flow of the gas that has escaped from the container that is to be treated and is diverted by the torispherical head onto the outside of the packaging means that is to be treated.

In other words, after the treatment gas has been emitted from the outlet aperture 420 of the treatment nozzle 42, the treatment gas enters the mouth area of the packaging means. This displaces the treatment gas that is already present in the packaging means, which flows back out of the mouth area of the packaging means and is diverted by the torispherical head of the treatment nozzle 42 onto the outside of the packaging means.

The sensors can be disposed in either the first sensor position A or the second sensor position B, or else in both sensor positions.

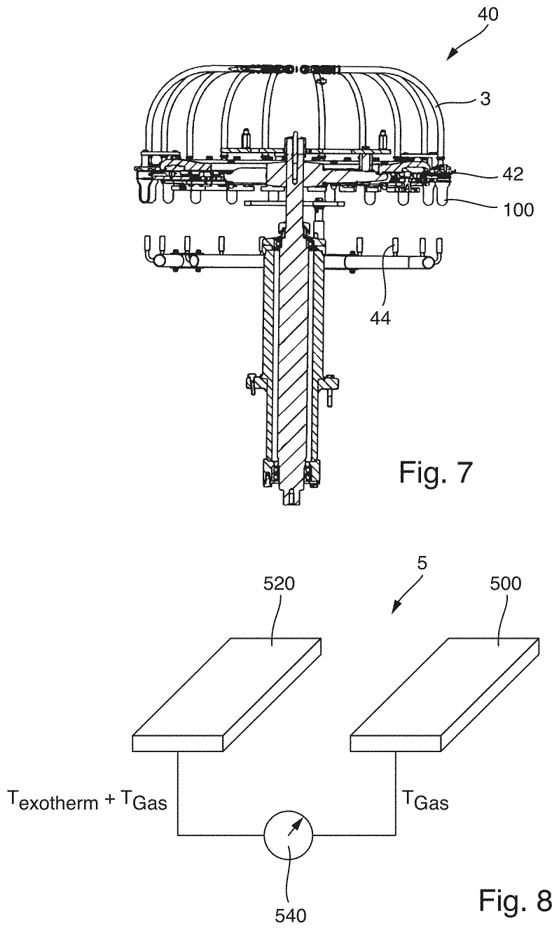

FIG. 7 shows a schematic sectional view through the treatment carousel 40, wherein the applicable treatment nozzles 42 for treating the packaging means 100 are equipped with nozzles that are designed as in FIG. 6, having sensors disposed at both sensor positions A and B.

Below the rotating treatment carousel 40, stationary treatment nozzles 44 are provided, on which it is again possible to provide a sensor for measuring the concentration.

FIG. 8 shows schematically the design of the sensor 5 in a preferred variant. The sensor 5 has a first passive surface 500 and a second surface 520 which is coated with a catalyst.

The passive surface 500 is accordingly chemically inert with respect to the treatment gas, and is in particular inert with respect to the hydrogen peroxide. The surface coated with a catalyst 520, however, causes an exothermic decomposition reaction of the hydrogen peroxide, and can be designed for example as a porous MnO₂ surface. Due to the exothermic reaction, the surface coated with a catalyst 520 is at a temperature which corresponds to the gas temperature T_{Gas} plus the heat T_{Exotherm} generated by the exothermic reaction. The passive surface 500, however, adopts only the gas temperature T_{Gas}. From the difference in temperature, which is determined by means of a temperature difference measurement device 540, the concentration of hydrogen peroxide in the flow of treatment gas that passes over the sensor 5 can be derived.

To the extent applicable, all individual features that are described in the example embodiments can be combined with each other and/or exchanged, without departing from the field of the invention.

### LIST OF REFERENCE SKINS

1 device
1 100 packaging means
2 vaporization device
20 carrier gas supply line
22 regulating valve
24 mass flow sensor
26 hydrogen peroxide supply line
28 regulating valve
3 treatment gas supply line
30 rotary distributor
4 isolator
40 treatment carousel
42 treatment nozzle
420 outlet aperture
44 stationary treatment nozzle
5 sensor
50 control cable
52 slip ring transmitter
54 RFID antenna
56 evaluation device
500 passive surface
520 surface coated with a catalyst
540 temperature difference measurement device
A first sensor position
B second sensor position

1. A device for treating system components and/or packaging means in a filling product filling system, the device comprising:
a vaporization device configured to provide a treatment gas flow containing vaporized hydrogen peroxide;
a treatment nozzle coupled to the vaporization device and configured to apply the treatment gas flow to the system components and/or the packaging means; and
a sensor configured to measure a concentration of the vaporized hydrogen peroxide in the treatment gas flow exiting the treatment nozzle.

2. The device according to claim 1, wherein the sensor is disposed in the treatment nozzle or on the treatment nozzle.

3. The device according to claim 1, further comprising a rotating structure upon which the sensor is disposed.

4. The device according to claim 3, further comprising a stationary structure,
wherein the sensor generates a signal relating to the vaporized hydrogen peroxide concentration as measured by the sensor and
the signal is transmitted by wire from the rotating structure of the device to the stationary structure of the device.

5. The device according to claim 3, further comprising a stationary structure,
wherein the sensor generates a signal relating to the vaporized hydrogen peroxide concentration as measured by the sensor and
the signal is transmitted wirelessly from the rotating structure of the device to the stationary structure of the device.

6. The device according to claim 1, wherein
the sensor generates a signal relating to the vaporized hydrogen peroxide concentration as measured by the sensor,
the vaporization device is configured to generate the treatment gas flow containing vaporized hydrogen peroxide by vaporizing hydrogen peroxide in a liquid phase supplied to the vaporization device, and
the supply of hydrogen peroxide in the liquid phase supplied into the vaporization device is regulated based on the signal from the sensor.

7. The device according to claim 1, wherein the sensor is a catalytic sensor configured to measure the vaporized hydrogen peroxide concentration via a temperature difference from a surface that is inert with respect to hydrogen peroxide.

8. The device according to claim 1, wherein the treatment nozzle comprises:
an outlet aperture configured to guide the treatment gas flow into a mouth area of the packaging means; and
a torispherical head configured to deflect displaced treatment gas that flows out of the mouth area of the packaging means onto an exterior of the packaging means.

9. The device according to claim 1, wherein
the sensor is a first sensor and is disposed in the treatment nozzle, and
the device further comprises a second sensor configured to measure the vaporized hydrogen peroxide concentration in the treatment gas flow exiting the treatment nozzle that is disposed on the treatment nozzle.

10. The device according to claim 3, wherein the rotating structure of the device is a treatment carousel upon which the treatment nozzle is disposed.

11. The device according to claim 4, wherein the signal is transmitted by wire from the rotating structure of the device to the stationary structure of the device by a slip ring transmitter.

12. The device according to claim 5, wherein the sensor comprises an RFID chip and the stationary structure includes an RFID antenna and the RFID chip is configured to transmit the signal from the sensor to the RFID antenna.

13. The device according to claim 4, wherein
the vaporization device is configured to generate the treatment gas flow containing vaporized hydrogen peroxide by vaporizing hydrogen peroxide in a liquid phase supplied to the vaporization device, and
the supply of hydrogen peroxide in the liquid phase supplied into the vaporization device is regulated based on the signal from the sensor.

14. The device according to claim 5, wherein
the vaporization device is configured to generate the treatment gas flow containing vaporized hydrogen peroxide by vaporizing hydrogen peroxide in a liquid phase supplied to the vaporization device, and
the supply of hydrogen peroxide in the liquid phase supplied into the vaporization device is regulated based on the signal from the sensor.

15. The device according to claim 8, wherein
the sensor is a first sensor that is disposed in a first sensor position in a treatment gas supply line immediately before the outlet aperture, and
the device further comprises a second sensor that is disposed in a second sensor position in the torispherical head.

16. The device according to claim 3, wherein the treatment nozzle is disposed on the rotating structure and the treatment nozzle comprises:
an outlet aperture configured to guide the treatment gas flow into a mouth area of the packaging means; and
a torispherical head configured to deflect displaced treatment gas that flows out of the mouth area of the packaging means onto an exterior of the packaging means.

17. The device according to claim 16, wherein
the sensor is disposed in a first sensor position in a treatment gas supply line immediately before the outlet aperture, and
the device further comprises a second sensor that is disposed in a second sensor position in the torispherical head.

18. The device according to claim 16, wherein
the sensor generates a signal relating to the vaporized hydrogen peroxide concentration as measured by the sensor,
the vaporization device is configured to generate the treatment gas flow containing vaporized hydrogen peroxide by vaporizing hydrogen peroxide in a liquid phase supplied to the vaporization device, and
the supply of hydrogen peroxide in the liquid phase supplied into the vaporization device is regulated based on the signal from the sensor.

19. The device according to claim 18, wherein
the sensor is disposed in a first sensor position in a treatment gas supply line immediately before the outlet aperture and
the device further comprises a second sensor that is disposed in a second sensor position in the torispherical head, the second sensor generates a second signal relating to the vaporized hydrogen peroxide concentration as measured by the second sensor, and
the supply of hydrogen peroxide in the liquid phase supplied into the vaporization device is regulated based on the signal and the second signal.

20. The device according to claim 19, further comprising a stationary structure that includes the vaporization device,
wherein the signal and the second signal are transmitted from the sensor and the second sensor, respectively, of the rotating structure to the vaporization device of the stationary structure.
